(19) United States
(12) Patent Application Publication
(10) Pub. No.: US 2004/0009195 A1
(43) Pub. Date: Jan. 15, 2004

(54) Modified sialic acid vaccines

(76) Inventors: Harold J Jennings, Gloucester (CA); Wei Zou, Gloucester (CA); Silvia Borelli, Ottawa (CA)

Correspondence Address: NATIONAL RESEARCH COUNCIL OF CANADA, 1500 MONTREAL ROAD, BLDG M-58, ROOM EG12, OTTAWA, ONTARIO, K1A 0R6, CA

(21) Appl. No.: 10/343,039

(22) PCT Filed: Jul. 26, 2001
(86) PCT No.: PCT/CA01/01080

(51) Int. Cl. A61K 39/02; A61K 31/739

(52) U.S. Cl. 424/234.1; 514/54

(57) ABSTRACT

The sialic acid component of a sialic acid unit-containing cell surface marker characteristic of cancerous mammalian cells is modified, so that cells normally expressing such a marker express instead a modified sialic acid unit-containing cell surface marker which is strongly immunogenic. For example, the present invention enables, in a portion of patient cells which regularly express GD3 (i.e. various types of cancer cells), the expression of a highly immunogenic surface antigen namely, GD3 in which the sialic acid residues are modified. The modification is suitably N-acylation of a precursor of the sialic acid, so that the N-acylated precursor becomes chemically incorporated in the sialic acid during its intracellular biochemical synthesis. Antibodies specific for the modified antigen, which can be induced using a conjugate of a suitable portion of the modified sialic acid unit-containing marker and a carrier, can then be used to eliminate cells which express the modified GD3. Vaccines can be prepared utilizing conjugates of the modified sialic acid-containing marker, or utilizing antibodies produced in response to exposure of a suitable subject to the modified sialic acid-containing marker, for managing cancer conditions which involve cancer cells characterized, at least in part, by expression of modified sialic acid unit-containing marker.

This invention relates to the field of medical treatments and therapeutic compositions for use therein. More specifically, it relates to methods and compositions for treatment and prophylaxis of cancer in human patients.

Despite the very extensive research efforts and expenditures over recent years, cancer remains one of the most life threatening diseases in the world. Cancer therapy remains very difficult. Scientists have long been exploring the possibility of developing vaccines for the treatment and prevention of cancer in human patients. Although this approach has been viewed as the therapy of the future, progress has been modest and the incidence of clinical failure has been very high.

Creating cancer vaccines is problematic, due largely to the fact that patients fail to mount an effective immune response to cancerous cells, because cancer cells generally fail to produce immunogenic markers that sufficiently distinguish them from normal cells. Although the patterns of cell surface carbohydrate antigens of cancer cells differ from those of normal cells, the individual structures of their antigens are identical.

Despite the structural identity of individual antigens found on normal cells and cancer cells, attempts have been made to exploit cancer cell carbohydrate antigens as potential cancer vaccines. The observation that specific antigens are overexpressed by certain tumour types has enabled the development of simple monovalent antigen vaccines against various tumour types. It has also been observed that, in animals and humans, provided that the carbohydrate antigens are conjugated to a protein carrier, the resulting conjugate vaccines can be sometimes used to raise antibodies that are specific for each carbohydrate antigen.

However, the antibodies so induced are usually of low titer and poor endurance (mostly IgM). Despite this drawback, they are used, on the basis that, after surgical or chemical treatment of cancer, the antibody levels will remain sufficiently high, during a short convalescence period, to dispose of any remaining cancer cells.

Clinical trials using some of these carbohydrate antigen-protein conjugate vaccines have demonstrated that they can increase remission times in some patients. However, their use as therapeutic agents is far from satisfactory.

Dramatic changes in gangliosides have been noted in cancer cells of neuroectodermal origin (for example, melanoma, neuroblastoma, glioma and astrocytoma) and a few other tumour types (e.g. small cell lung cancers and sarcomas). These changes are sufficiently prominent that attempts have been made to use these gangliosides as target antigens for the immunodiagnosis and immunotherapy of these cancers. Both anti-ganglioside monoclonal antibodies and ganglioside vaccines have been explored for the therapy of cancer. Most of the studies in this area have been on malignant melanoma and neuroblastoma

GD3 is one ganglioside which is highly expressed on the surface of a variety of cancer cells but is not significantly expressed in most normal tissues. Although certain ganglioside-based vaccines to melanoma have been tested (Int. J Cancer, 1985, 35, 607-612, J Clin. Oncol. 1994, 12, 1036-1044), results have not been entirely satisfactory and have failed to provide an adequate GD3 vaccine. GD3 appears to be poorly immunogenic in humans, and the focus thus far has been on more immunogenic gangliosides and particularly GM2 and GD2.

As mentioned above, GD3 is poorly immunogenic in humans. Antibody-directed therapy is presently being modified by combining antibodies with cytokines, by the use of humanized antibodies and by the development of anti-idiotype antibody vaccines. Nonetheless, progress has been limited. Attempts to chemically modify gangliosides by making the lactone, amide and O-acetylation to augment their immunogenicity have thus far failed to provide a means of raising anti-GD3 antibodies which react with melanoma cells (Ragupathi, (1996) Cancer Immunol. Immunother. 43:152).

Apart entirely from the field of cancer treatment, in the field of infectious disease control vaccine compositions based on chemically modified meningococcal polysaccharides, and their use for immunizing mammals against Neisseria meningitidis and E. coli K1, has been reported (U.S. Pat. No. 5,811,102). A modified B polysaccharide of N. meningitidis is prepared chemically from the polysaccharide isolated from N. meningitidis. The modified polysaccharide has sialic acid residue N-acetyl groups (C₂) replaced by a saturated or unsaturated C₃₋₅ acyl group. This modified polysaccharide is conjugated to an immunologically suitable protein to produce a conjugate of enhanced immunogenicity. A mammal may be immunized with the vaccine composition, to induce a specific immune response in the animal suitable to provide active protection from N. meningitidis infection. Alternatively, blood may be collected and the gamma globulin fraction maybe separated from the immune serum, to provide a fraction for administration to a suitable subject to provide passive protection against or to treat on-going infection caused by these. microorganisms. However, to date no relevance of this remote field to cancer treatment has been taught or suggested.

It is thus an object of the present invention to provide novel compositions suitable for use as anti-cancer vaccines and, further, a process for enhancing the specific immunogenicity of mammalian cancer cells, and exploiting this enhanced immunogenicity in a vaccination approach to the management of cancer in human patients.

Bioengineered Cells

One challenge overcome by the present invention is the poor immunogenicity of GD3. GD3's poor immunogenicity is believed to be due to the fact that cancer cells fail to produce strong immunogenic markers that sufficiently distinguish them from normal cells. The present invention overcomes this problem by providing a method of bioengineering tumour cells to express unnatural GD3 antigens, in which the sialic acid residues are chemically modified, on the cell surface. Expression of such modified GD3 antigen makes the tumour cells vulnerable to immune attack from antibodies, which can be generated using correspondingly modified GD3 glycoconjugates.

GD3 is known to be expressed on the surface of melanoma, neuroblastoma, sarcoma and lung cancer cells. Other cancerous or otherwise diseased cell types are suspected to express GD3, and cells can be screened for GD3 expression using standard techniques known in the art.

N-acyl modified disialolactoside-carrier conjugates and specific antibodies raised using these conjugates which are not cross-reactive to normal cell surface GD3 have been provided. Incubation of GD3 expressing cancer cells with respectively modified precursor in GD3 synthesis in vivo causes GD3 on the cell surface to incorporate the modified precursor and produce modified GD3, which renders these cells recognizable to the specific antibodies raised and therefore susceptible to the antibody-depended cytolysis. Since the expression of modified GD3 can be regulated by the administration of the modified precursor, and is critical to the cytolysis, the immune response in vivo may be controlled to reduce the risk of inducing an unwanted long-term auto immune response.

In one aspect of the invention there is provided a process of enhancing the specific immunogenicity of viable, proliferating mammalian cancer cells to levels sufficient to allow the effective recognition and destruction of such cells by an immuno-response in vivo. This process comprises providing to said cells a chemically modified precursor of a suitable sialic acid unit-containing cell surface marker capable of rendering said cancer cells immunologically distinctive from related, normal cells; causing biochemical incorporation of said modified precursor into the sialic acid unit-containing cell surface marker during intracellular synthetic processes; and eventual surface expression of the sialic acid unit-containing surface marker incorporating said modified precursor in a form capable of eliciting said level of immune response.

In another embodiment of the invention, there is provided a process of enhancing the specific immunogenicity of viable, proliferating mammalian cancer cells to levels sufficient to allow the effective recognition and destruction of such cells by an immuno-response in vivo, wherein the process comprises providing to said cells a chemically modified precursor of GD3; causing biochemical incorporation of said modified precursor into GD3 during intracellular synthetic processes; and eventual surface expression of GD3 incorporating said modified precursor in a form capable of eliciting said level of immune response.

In another embodiment of the invention there is provided a method of immunogenic marking and targeting of mammalian cancer cells. The cells have GD3 cell surface markers incorporating modified precursors capable of initiating an immune response in a mammalian system containing them which is sufficiently strong to effectively combat the proliferation of such cells.

In another embodiment of the invention, there is provided a conjugate of a modified GD3 incorporating N-acylated sialic acid units and a carrier and the use of this conjugate in the preparation of a vaccine for managing cancer conditions in mammalian patients. Preferably the N-acylation is a C₃ to C₈ alkyl or alkyl-aromatic group. For example, N-propionyl GD3 (GD3 Pr), N-butyril GD3 (GD3 Bu) and N-benzoyl GD3 (GD3 Bz) are among the N-acyl GD3s contemplated. A person skilled in the art, in light of the disclosure herein, could routinely identify and synthesize suitable N-acyl GD3s and determine appropriately modified precursors for use to induce expression of the modified GD3 on the cancer cell surface. Any suitable carrier may be conjugated to the N-acylated GD3 to form the conjugate. The carrier is preferably a protein. In some instances, it will be desirable to use a carrier selected from KLH, Tetanus toxoid and bacterial outer membrane proteins.

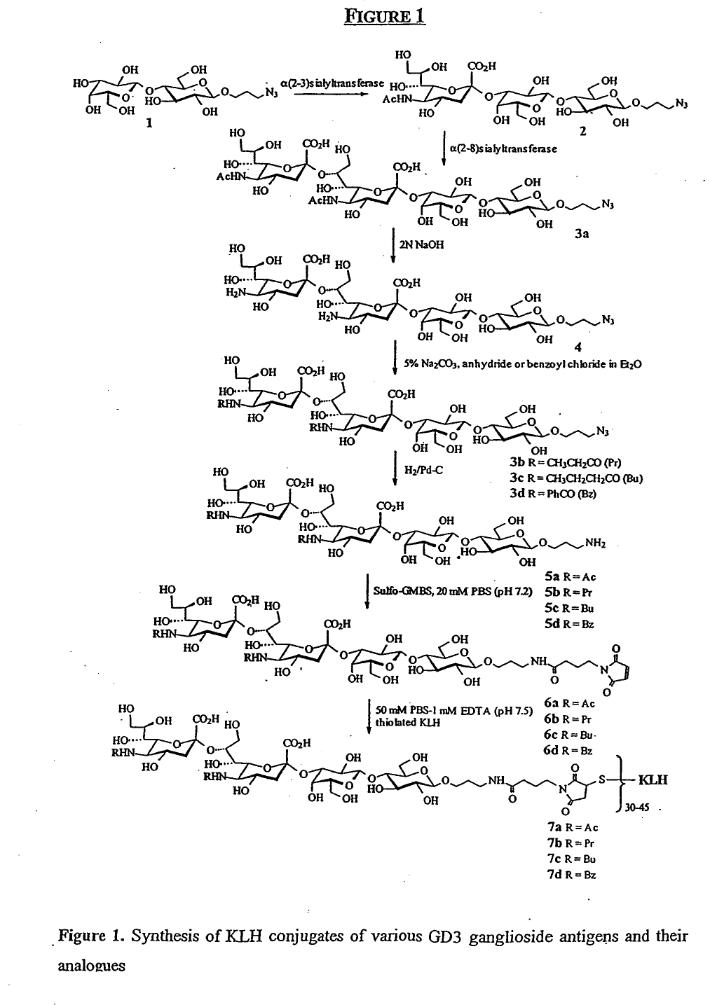

FIG. 1 is a depiction of the major steps in an embodiment of the synthesis of KLH conjugates described in Examples 1 and 2.

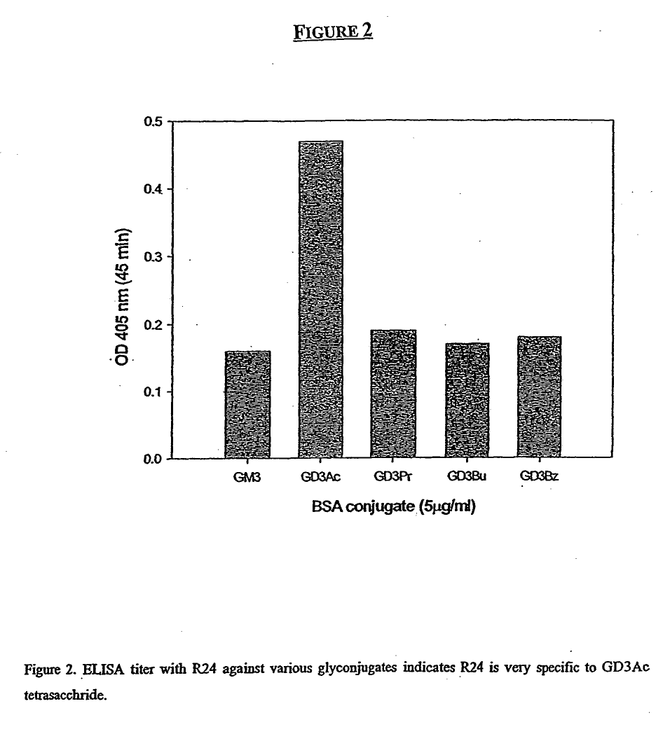

FIG. 2 is a graphical depiction of the reaction of antibody R24 to various glyconjugates.

In vitro, cancer cells incorporate modified mannosamine precursors and strongly express modified cell surface GD3 within 24 hours of exposure to the modified, precursor. Compared to polysialic acid antigens the complete metabolic turn over of GD3 is very slow. After 10 days cells still express modified GD3.

In therapeutic applications, the patient preferably receives modified precursor several times per week, with each total weekly dose preferably being in the range of 2 to 20 g, more preferably between 5 and 15 g and even more preferably between 8 and 12 g (based on a 60 to 70 kg patient). In some instances, daily precursor doses in the range of 10 to 40 mg/kg body weight will be desirable.

Preferably, cancer cells are recovered from the patient between 5 to 10 days after commencing treatment and expression of modified GD3 on the cell surface is determined by standard means such as immune-staining and flow cytometry. Preferably, where modified GD3 accounts for less than 10% of the total GD3 expressed on the surface of the cancer cells from the patient, the weekly precursor dose is increased. More preferably, the expression of modified GD3 accounts for at least 50% of the total GD3 expressed on the cancer cell surface, and weekly precursor dosage will be increased until at least this level of expression is observed. Where the patient's condition permits the administration of higher levels of modified precursor, it may be desirable to increase the weekly precursor dosage until modified GD3 accounts for at least 90% of the total GD3 expressed on the cell surface. Expression of modified GD3 in patient cells may be compared to expression levels obtained in cells cultured in vitro in the presence of the precursor. It is within the capacity of a skilled technician, in light of the disclosure herein, to determine a suitable dose and administration frequency for a given patient.

The modified precursor is preferably an N-acylated mannosamine. More preferably, the precursor is an N-acylated mannosamine N-acylated with a C₃ to C₈ alkyl or alkyl-aromatic group. Yet more preferably the precursor is selected from N-propionyl mannosamine, N-butyril mannosamine and N-benzoyl mannosamine. In some instances it may be desirable to administer a combination of precursors.

Antibodies specific for the modified GD3 maybe administered to the patient and/or, where the patient is not significantly immunocompromised, these antibodies may be generated in the patient in response to specific antigens.

Where exogenous antibodies are to be used, these antibodies may be produced by any suitable means. These antibodies are preferably humanized monoclonal antibodies produced according to standard methods known in the art.

Humanized exogenous antibodies specific for the modified GD3 of interest are preferably administered at regular intervals during the period of modified precursor administration. Antibodies are preferably administered by daily injection. A variety of injection methods are contemplated (e.g. intramuscular, intraperitoneal, intravenous). Preferably the antibody is injected either intravenously for circulation throughout the body (particularly useful for the control of metastasis) and/or where there is a solid tumour, near the tumour site.

The dose of exogenous antibody may be determined with reference to cancer cell proliferation or tumour size. The total daily dose of exogenous antibody recognizing modified GD3 is preferably between 100 μg and 5 mg per day. Where tumour size assessment is feasible, it is preferable to use an antibody dose in the range of between 5 mg to 500 mg per square meter of total tumor surface area. It will be apparent that a suitable dose for a particular patient can be readily determined in light of the disclosure herein, together with the patent's weight and condition. In particular, the sufficiency of a particular dose can be determined routinely by culturing SK-Mel-28 cells in the presence of complement and substantially undiluted patient serum (obtained after at least 5 days of treatment). Complement dependant cytolysis of at least 50% of the SK-Mel-28 cells indicates that the antibody dose is sufficient. Lower levels of cytolysis indicate a higher antibody dose should be used.

The conjugate is preferably administered in a series of at least 3 vaccinations over the course of at least 3 weeks. The dose administered at each vaccination is preferably between 5 and 500 μg disialolactoside per patient, more preferably between 10 and 100 μg disialolactoside per patient. The glycoconjugate maybe delivered by any pharmaceutically acceptable means, but is preferably delivered together with an immuno-adjuvant in a pharmaceutically acceptable carrier.

The precise dose and administration schedule for a particular patient can be readily determined in light of the disclosure herein, and the patient's existing titer of antibodies recognizing modified GD3. This titer can be determined by methods known in the art. An IgG titer below that equivalent to 1500 on Table 1 indicates that further vaccination is required.

In cases where the patient's condition or wishes preclude administration of the modified GD3-protein conjugate or antibodies to the modified GD3, it is possible to simply administer the modified precursor, thereby causing expression of modified GD3 on the surface of cancer cells. If the patient is not significantly immunocompromised, an immune response to the modified GD3 will eventually occur, and can provide some therapeutic benefit to the patient.

Thus, the present invention provides a selective immunotherapy method which reduces the risk of an unwanted autoimmune response. The present invention provides antibodies which will not ordinarily react significantly with normal tissues because no modified GD3 antigen is normally found in mammals. However, these antibodies will recognize cell surface GD3 antigens incorporating corresponding modified precursor. Such modification can be achieved by intervening in the biosynthetic pathway of GD3 by administering a precursor of GD3. The biosynthetic pathway of GD3 is known. Thus, in light of the disclosure herein, a person skilled in the art could readily identify suitable GD3 precursors for modification. The combination of vaccine and precursor may effectively stimulate the immune response in a controlled way and cancer cells expressing such modified GD3 may be eliminated.

In some cases it may be desirable to treat a patient with an antibody raised against a modified GD3 but known to cross react with native GD3 on cancer cells. As previously described, normal tissues generally do not express GD3. Thus, where an antibody will cross react to native GD3, it may be useful in immunotherapy. While it is believed that a stronger immune response will generally be seen to modified GD3, there may be situations where it is not possible to administer the modified precursor to a patient, or where the GD3 on the surface of target cells cycles very slowly, reducing the rate of precursor incorporation into cell surface GD3. In such cases, an antibody cross-reactive to native GD3 may be used to lead an immune attack on the diseased cells.

### EXAMPLES

### Example 1

### Synthesis of Various GD3 Ganglioside Antigens and Their Analogues

Reference numerals refer to corresponding chemical moieties shown in FIG. 1.

3-Azidopropyl GD3 Tetrasaccharide (3a)

To a solution of 3-azidopropyl lactoside (1) (200 mg) in 50 mM Tris (pH 7, 20 mL) with cytidine 5′-monophospho-N-acetylneuraminic acid (“CMP-Neu5Ac”) (50 mM) and MgCl₂ (20 mM) was added a-2,3-sialyltransferase (10 units). The mixture was adjusted to pH 7 and incubated for 5 h at 32° C. Centrifuge at 15,000 rpm for 30 min to remove insoluble material. To above solution (approx. 20 mL) was added CMP-Neu5Ac (25 mM) and MgCl₂(10 mM) to a volume about 30 mL. a-2,8-Sialyltransferase (10 units) was added and the mixture was incubated for 3 h at 37° C. Centrifuge at 15,000 rpm for 30 min to remove insoluble material. The resulting solution was lyophilized and further purified by a biogel P-6 column using 0.03 M NH₄HCO₃ as eluent to afford GD3 tetrasaccharide (3a) (210 mg) and GM3 trisaccharide (2) (45 mg). N-deacetylation of GD3 tetrasaccharide (4)

A solution of 3a in 2 N NaOH (10 mg/mL) was heated at 100° C. for 4 h. Upon cooling the solution was carefully neutralized by addition of 2 N HCl, and purified by passage through a Biogel P-6 column, using 0.03 M NH₄HCO₃ as eluent. The product was obtained after lyophilization as an amorphous solid 4 in quantitative yield.

N-Acylation of N-deacetylated GD3 Tetrasaccharide (3b, 3c, 3d)

Four disialolactosides were synthesized, namely N-propionyl (GD3Pr), N-butyril (GD3Bu), N-Benzoyl (GD3Bz) and N-acetyl (GD3Ac).

To a solution of 4 (5 mg) in 5% NA₂CO₃ (2.5 mL) was added propionic anhydride (10 uL×3 with 10 min interval) at room temperature with vigorously stirring. After 30 min the mixture was adjusted to pH 11.0 by the addition of 2 N NaOH and kept for 1 h. The solution was then adjusted to pH 8.0 by 0.5N HCl, and purification was achieved by passing through a Sephadex G-10 column, using water as eluent. The product 3b was obtained after lyophilization as an amorphous solid in quantitative yield.

To a solution of 4 (5 mg) in a mixture of 5% Na₂CO₃ (2.5 mL) and diethyl ether (2.5 mL) was added butyric anhydride (30 μL) or benzoyl chloride (30 uL) at room temperature with vigorously stirring. After 30 min the organic layer was removed and the aqueous solution was adjusted to pH 11.0 by the addition of 2 N NaOH and kept for 1 h. The solution was then adjusted to pH 8.0 by 0.5N HCl, and passed through a Sephadex G-10 column, using water as eluent. The products 3c and 3d were obtained after lyophilization as an amorphous solid, respectively, in quantitative yield.

Reduction of Azido Group to Amine (5a-5d) and Introduction of Maleimide (6a-6d)

A solution of above compound (3a-3d) (5 mg) in water (0.5 ml) was subjected to catalytic (Pd/C) hydrogenation (30 p.s.i.) for 2 h, respectively. The filtrate was passed through a Sephadex G-10 column, using water as eluent. The lyophilized products (5a-5d) were dissolved in 20 mM PBS (2 ML, pH 7.2) and mixed with Sulfo-GMBS (5 mg). The solution was kept at room temperature for 0.5 h, when TLC (CHCl₃-MeOH-H₂O 9:9:1) indicated the reaction was complete with the formation of a faster moving product. Purification on a Sephadex G-10 column, eluted with water, gave the products (6a-6d) in quantitative yield as an amorphous solid after lyophilization.

### Example 2

### Conjugation to KLH

A solution of thiolated Keyhole limpet hemocyanin (“KLH”) (3 mg) in 50 mM PBS buffer with 1 mM EDTA (pH 7.5, 1 mL) was mixed with the maleimide-containing carbohydrates (6a-6d) (3-4 mg) prepared above. The reaction mixture was incubated at room temperature for 6 h. Purification on a Biogel A 0.5 column (1.6×30 cm), eluted with 0.02 M PBS buffer with 50 mM NaCl (pH 7.1), gave the respective conjugates (7a-7d) in a volume about 6-7 mL. Sialic acid content was assayed using the resorcinol method and the BCE protein assay revealed that each KLH molecule carries about 30-45 GD3 tetrasaccharide chains.

### Example 3

### Immunization and Antibody Production

3a - Immunization and Antibody Detection

Groups of female BALB/c mice, 6 to 8 weeks of age, were immunized intraperitoneally with KLH glycoconjugate. Each mouse in-groups of 10 was injected with 2 μg of saccharide in 0.10-0.15 ml PBS buffer with monophosphoryl lipid A (“MPL”) (2.0 μg). 5 mice in a control group were injected with same volume of PBS buffer. The mice were boosted on day 7, 14, and 21. The mice were bled on day 0, 7, 14, 21, with a final bleeding on day 31. ELISA was used to detect antibodies according to standard procedures. Cells producing antibodies specific for the KIM glycoconjugate were identified, isolated and further screened by standard means.

The results were summarized in Table 1. All four conjugates are immunogenic and gave high titer of antibodies. GD3Bu-KLH conjugate is most immunogenic, followed by GD3Pr-KLH, GD3Ac-KLH and GD3Bz-KLH. The extension of N-acyl chain seemingly correlates to the increased immunogenicity.

Antiserum of GD3Bu and GD3Bz conjugates shows high specificity and no cross-reactivity to unmodified GD3 on the surface of certain cell types. Thus, these epitopes are likely very distinctive and particularly useful in cancer immunotherapy. Two parameters are considered in the use of metabolic precursor to remodel cell surface: the incorporation efficiency and the metabolic rate.

3b - Preparation of Monoclonal Antibodies

Two anti-GD3Bu monoclonal antibodies, one IgG1 and the other IgG2a were selected and established by ELISA and flow cytometry analysis. Both antibodies cross-react to GD3Pr on the cell surface but not GD3Ac (see Table 2). The relative binding affinity to GD3Pr and GD3Bu has not been determined, however, based on the flow cytometric analysis and the similar expression of GD3 analogs on cell surface IgG2a showed a similar affinity to both GD3Bu and GD3Pr, whereas IgG1 is more specific to GD3Bu.

The competitive inhibition experiment using disialolactoside confirms the epitope recognized by these Mabs is a modified GD3 tetrasaccharide. N-Acyl group of sialic acid residue is definitely involved in the binding, however, the detailed structural parameters are yet to be further defined.

The production of IgG1 and IgG2a is typical in a T-cell dependant immune response. IgG2b antibodies were also detected in polyclonal antiserum.

Cytotoxicity Assay—Complement Dependant Cytolysis

Following preculture with precursor (ManNBu) SK-Mel-28 cells were further treated with both mAbs (IgG1 and IgG2a) and incubated with rabbit complement. The tumour cell lysis is dependent on the concentration of mAbs (see Table 3). Both antibodies were effective in promoting cancer cell killing in vitro. Polyclonal antiserum is also very effective to kill modified SK-Mel-28 cells.

Cells incubated with the modified precursors for various periods of time at various dosages are harvested, rinsed in PBS, and cultured in the presence of suitable complement and an antibody specific for GD3 ganglioside analogues from Example 4, and cytotoxicity is assessed by standard means. Cells incubated under suitable conditions showed complement-mediated cell lysis of over 50% when incubated with complement and antibodies specific to GD3 ganglioside analogues.

### Example 4

### Induction of Expression of Modified Gangliosides In vitro

SK-Mel-28 human melanoma cells normally expressing GD3 were incubated with modified sialic acid precursors. The modified sialic acid precursors used were N-acylated Mannosamines(“ManNAc”), including: N-propionyl mannosamine (“ManNPr”), N-butyril mannosamine (“ManNBu”), and N-benzoyl mannosamine (“ManNBz”).

The reactivity and cross reactivity among the anti-sera and surface antigens of SK-Mel-28 was analysed (see Table 4). Cross reactivity between GD3Ac and GD3Pr antiserum, GD3Pr and GD3Bu antiserum was observed, but not between GD3Ac and GD3Bu antiserum, and GD3Ac and GD3Bz antiserum. Murine IgG3 antibody R24 is specific to the terminal NeuAc of disialolactoside and does not significantly cross react with other N-acyl derived analogs when assayed by ELISA (FIG. 2). This antibody is suitable for use in monitoring GD3Ac expression in flow cytometry assays.

The incorporation and metabolism of the surface GD3 antigen were also investigated. Precursors at 1 mg/ml concentration achieved good expression of modified GD3, respectively within 24 hours, and increased precursor concentration (3 and 5 mg/ml) did not add further expression. Modified GD3 on SK-Mel-28 cells in vitro was still found 10 days after removal of precursors from the growth medium, when two populations of antigens, unmodified and modified GD3 were detected by mAb R24 and respective antiserum.

The relative quantity of modified and unmodified GD3 expressed on the SK-Mel-28 was analysed by capillary electrophoresis-mass spectroscopy (“CE-MS”). The glycolipids extracted from cells grown in various concentrations of ManNBu were separated by capillary electrophoresis and negative charged glycolipids were detected. GD3 was the dominant ganglioside found in SK-Mel-28 cells. When ManNBu was added to the medium, the modified GD3 was biosynthesized and expressed. The relative expression of GD3 and its analog was then estimated by CE-MS, which was in agreement to the results observed in flow cytometry assay, i.e. modified GD3 is well expressed even at 1 mg/ml precursor concentration.

In vitro, cancer cells incorporate modified mannosamine precursors and strongly express modified cell surface GD3 within 24 hours of exposure to the modified precursor. Compared to polysialic acid antigens the complete metabolic turn over of GD3 is very slow. After 10 days the cells still express modified GD3, indicating that modified GD3 is valuable as an immunotarget.

The disialolactoside formed in the glycoconjugates appear to accurately imitate the epitope expressed on the cell surface. Unmodified GD3 was not significantly recognized by the antiserum. Thus, the present invention provides a selective immunotherapy method which reduces the risk of an unwanted autoimmune response. The antibodies of the present invention will not ordinarily react significantly with normal tissues because no modified GD3 antigen is normally found in mammals. However, these antibodies will recognize cell surface GD3 antigens incorporating corresponding modified sialic acid residues. Such modification can be achieved by intervene the biosynthetic pathway of GD3 by administrate ManNBu as precursor of the sialic acid. The combination of vaccine and precursor may effectively stimulate the immune response in a controlled way and cancer cells expressing such modified GD3 may be eliminated.

Thus, cancer cells take up modified precursors and incorporate them into GD3 on the cell surface in an immunogenic form and this can be used in the treatment of cancer and the prevention of metastasis.

### Example 5

### Induction of Expression of Modified Gangliosides In vivo

BALB/c nude Mice are inoculated with SK-Mel-28 human melanoma cells (10⁷ cells/mouse) and 5 days after inoculation the mice are treated once per day, 5 days a week for two weeks (by i.v. injection) with antibody specific for modified GD3 ganglioside analogue (200 μg, from Example 3), and a modified precursor (1,5 and 10 mg/mouse). As a control, one group of animals receives human IgG instead of the specific antibody from example 3. Tumour growth is routinely monitored by measurement of tumour size and calculation of tumour volume. In combination with modified precursor, the antibody specific for the modified GD3 can reduce tumour size when compared with a control group of mice.

### Example 6

### Control of Metastatic Cancer Cells

The experiments in mice are carried out as described in Example 5 except that in this case the spleens of the mice are analyzed for the presence of metastatic cells. On day 25, spleens are excised and cell suspensions prepared in medium RMPI 8% FEBS. One fifth of the aliquots from the individual mice are used to initiate serial two fold dilution in 24 well plates in 1 mL of RPMI 8% FBS. Cultures are fed regularly and monitored over a period of one month to score positive wells containing tumours. Spleen samples having tumour cells are scored positive and the samples that had no tumour cells at all dilutions are scored negative. Following cell cultures of the spleen cells, the metastatisized tumour cells are easily distinguished from the normal spleen cells, by microscopic examination. Fewer tumour cells are found in the spleen of the mice treated with a combination of the modified precursor and the antibody specific for the modified GD3 ganglioside analogue.

Thus, the metastasis of tumour cells can be controlled by modification of surface GD3 glycoconjugates using modified analogs and then applying immunotherapy based on antibodies specific for the modified antigen. These antibodies could be either passively administered as described herein, or induced in situ by direct immunization using an appropriate N-modified GD3—protein conjugate vaccine.

Thus, it will be appreciated that there have been provided novel compositions suitable for use as anti-cancer vaccines and, further, a process for enhancing the specific immunogenicity of mammalian cancer cells, and exploiting this enhanced immunogenicity in a vaccination approach to the management of cancer in human patients.

TABLE 1
Antibody titers by ELISA against BSA conjugate of GD3 analogs

| | GD3Ac- | | GD3Pr- | | GD3Bu- | | GD3Bz- | |
|---|---|---|---|---|---|---|---|---|
| Mouse | IgG | IgM | IgG | IgM | IgG | IgM | IgG | IgM |
| 1 | 3200 | 1600 | 400 | 200 | >12800 | 6400 | 800 | 800 |
| 2 | 800 | 200 | >12800 | 3200 | >12800 | 6400 | 800 | 3200 |
| 3 | 12800 | 1600 | 3200 | 800 | >12800 | 6400 | 800 | 100 |
| 4 | 6400 | 400 | 3200 | 12800 | >12800 | 1600 | 800 | 6400 |
| 5 | 3200 | 800 | 12800 | 3200 | >12800 | 1600 | 800 | 3200 |
| 6 | >12800 | 1600 | >12800 | 3200 | >12800 | 6400 | 800 | 1600 |
| 7 | 1600 | 1600 | >12800 | 3200 | >12800 | 1600 | 400 | 800 |
| 8 | 12800 | 1600 | >12800 | 12800 | >12800 | 1600 | 800 | 800 |
| 9 | 3200. | 1600 | 3200 | 3200 | >12800 | 200 | 800 | 800 |
| 10 | 6400 | 800 | >12800 | 1600 | >12800 | 800 | 800 | 400 |

a. Four.groups of mice (n = ) were immunized with GD3Ac-KLH, GD3Pr-KLH, GD3Bu-KLH, and GD3Bz-KLH glycoconjugates respectively.
b. The titers represent the highest dilution of serum (obtained on day 31) with an ODD ≧ 0.25 after 30 min.

TABLE 2
The specilicity of two monoclonal antibodies generated
from Balb/c mice after vaccination using GD3Bu-KLH conjugates

| mAb | GD3Ac-cell | GD3Pr-cell | GD3Bu-cell | GD3Bz-cell |
|---|---|---|---|---|
| IgG1 | − | + | ++ | − |
| IgG2a | − | ++ | ++ | − |

a. The cell surface GD3 analogs were obtained by biochemical engineering using N-acyl mannosamines as precursors.
b. (++) indicates large population of cells labeled by fluorescin in flow cytometric assay, (+) shows only minor binding to fluroscin, and (−) no binding.

TABLE 3
Antibody dependent complement-mediated cytotoxicity

| | IgG1 | | IgG2a | | GD3Bu antiserum |
|---|---|---|---|---|---|
| Concentration mg/ml | 0.25-1.0 | 0.02-0.10 | 0.25-1.0 | 0.02-0.10 | 10-40 dilution |
| % Cytolysis | 78-90 | 0-20 | 79-91 | 10-20 | 31-46 |

TABLE 4
Specificity and cross-reactivity of modified GD3 on SK-Mel-28
cell surface with antisera raised against modified and
unmodified GD3-KLH conjugates

| Antibody | Precursor | | | |
|---|---|---|---|---|
| or serum | ManNAc | ManNPr | ManNBu | ManNBz |
| R24 | ++ | + | + | + |
| Pab-Ac | ++ | + | + | + |
| Pab-Pr | + | ++ | + | − |
| Pab-Bu | − | + | ++ | + |
| Pab-Bz | − | − | + | ++ |

a. The cell surface GD3 analogs were obained by biochemical engineering using N-acyl mannosamines as precursors.
b. (++) indicates large population of cells labeled by fluorescin a flow cytometric assay, (+) shows only minoc binding to fluroscin, and (−) no binding.

What is claimed is:

1. A process of enhancing the specific immunogenicity of viable, proliferating mammalian cancer cells which express a GD3 cell surface marker to levels sufficient to allow the effective recognition and destruction of such cells by an immuno-response in vivo, which comprises providing to said cells a chemically modified precursor of the GD3 cell surface marker capable of rendering said cancer cells immunologically distinctive from related, normal cells; causing biochemical incorporation of said modified precursor into the GD3 cell surface marker during intracellular synthetic processes; and eventual surface expression of the GD3 cell surface marker incorporating said modified precursor in a form capable of eliciting said level of immune response.

2. The process of claim 1 wherein the chemically modified precursor is an N-acylated precursor.

3. The process of claim 2 wherein the N-acylated precursor is N-acylated by a C₃ to C₈ alkyl or alkyl-aromatic group.

4. The process of claim 1 wherein the chemically modified precursor is an N-acylated mannosamine.

5. The process of claim 1 wherein the precursor is N-propionyl-mannosamine.

6. The process of claim 1 wherein the precursor is N-butyril mannosamine.

7. The process of claim 1 wherein the precursor is N-benzoyl mannosamine.

8. A conjugate of a modified GD3 incorporating N-acylated sialic acid units and a carrier.

9. The conjugate of claim 8 wherein the carrier is a protein.

10. Use of the conjugate of claim 8 in the preparation of vaccine for managing cancer conditions in mammalian patients.

11. A process of reducing the viability of GD3 expressing cells in a patient comprising administering to the patient a composition including an antibody raised against and capable of reacting with an N-acylated GD3 and having cross-reactivity with unmodified GD3.

12. A process of reducing the viability of GD3 expressing cells in a patient comprising:
(a) administering to the patient a composition including an antibody raised against and capable of reacting with an N-acylated GD3; and,
(b) administering a GD3 precursor having the same N-acylation as the GD3 used to raise the antibody to the patient substantially together with the antibody.

13. The process of either one of claims 11 or 12 wherein the GD3 precursor is N-acylated with a C₃ to C₈ alkyl or alkyl-aromatic group.

14. The process of either one of claims 11 or 12 wherein the GD3 precursor is selected from the group consisting of: N-propionyl mannosamine, N-butyril mannosamine, and N-benzoyl mannosamine.

15. A method of inducing an immune response to a GD3 cell surface molecule in a mammalian subject comprising administering to the subject a conjugate of an immunogenic cell surface portion of an N-acylated GD3 molecule and a protein.

16. The method of claim 15 wherein the N-acylated GD3 molecule is N-acylated with a C₃ to C₈ alkyl or alkyl-aromatic group.

17. The method of claim 16 wherein the N-acylated GD3 molecule is-selected from the group consisting of: N-propionyl GD3, N-butyril GD3, and N-benzoyl GD3.
